(19) United States
(12) Patent Application Publication
(10) Pub. No.: US 2024/0306993 A1
(43) Pub. Date: Sep. 19, 2024

(54) RECONFIGURABLE WEARABLE HEALTH MONITORING DEVICE

(71) Applicant: KNOW LABS, INC., Seattle, WA (US)

(72) Inventor: JOHN CRONIN, Seattle, WA (US)

(21) Appl. No.: 18/608,478

(22) Filed: Mar. 18, 2024

Related U.S. Application Data
(60) Provisional application No. 63/490,647, filed on Mar. 16, 2023.

(51) Int. Cl. A61B 5/00; A61B 5/05; G04G 21/02

(52) U.S. Cl. CPC A61B 5/681; A61B 5/05; A61B 5/7225; G04G 21/025

(57) ABSTRACT

A reconfigurable wearable health monitoring device detachably attached to a smartwatch to be worn by a user. The device includes a radio frequency (RF) sensing module having at least one transmitting antenna configured to transmit radio frequency signals underneath the skin surface of the user; at least one receiving antenna configured to receive reflected low-power radio frequency signals; and a semiconductor chip configured to convert the low power radio frequency signals into high power radio frequency signals. A processor is configured to convert the digital signals into output information. A wireless communication module coupled with the processor is configured to transmit the output information to the smartwatch to reflect the user's health parameters. An alignment feature is configured with a rotating disc, an array, or a slider array to align the transmitting antenna and receiving antenna over a target vein without moving the smartwatch.

### CROSS-REFERENCE TO RELATED APPLICATIONS

This application claims priority to U.S. Provisional Application No. 63/490,647, filed on Mar. 16, 2023, the disclosure of which is incorporated herein by reference in its entirety.

### FIELD OF THE DISCLOSURE

The present disclosure is generally related to a health monitoring device and, more particularly, to a reconfigurable wearable health monitoring device mounted along with an electronic device configured to determine the health parameters of a user without moving the electronic device.

### BACKGROUND

The subject matter discussed in the background section should not be assumed to be prior art merely due to its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Frequent monitoring of health parameters is necessary for the modern urban and working population. Typically, doctors and patients have used a number of invasive and non-invasive procedures to measure vital signs such as blood sugar, heart rate, and temperature. To determine a blood glucose level, a blood sample is taken by pricking a spot on a patient's body, and the blood glucose level is tested using microscopic techniques. Although patients reliably sample their blood glucose levels using these procedures, due to the requirement to puncture the skin, not everyone is ready to submit to these tests regularly. Furthermore, there is always the possibility of introducing potentially harmful microorganisms into a patient's body due to these intrusive treatments.

Alternatively, a non-invasive technique is used in the form of a device that can accurately measure the glucose content inside a user's blood. The device is equipped with transmitting and receiving antennas that emit and receive radio signals that reflect from blood within the person. The reflected signals are processed to determine the glucose content. Generally, these devices are additionally integrated into wristwatches, such as multimedia tools, smartwatches, or smart bands.

Moreover, multiple health monitoring devices, such as smartwatches and smart bands, are used. These bands and watches are integrated with sensors that are capable of measuring the health parameters of the user within a quick span of time. In order to measure these parameters accurately, these devices are required to be placed exactly over a target area. The user is required to be well known about the target location over his/her body where the device is required to be kept. Since the sensor integrated into these devices require a specific location to work upon and draw accurate results. However, due to the interest of obtaining accurate results, the user is required to constantly place or move these devices all over the body, making it difficult to measure the health parameters. Further, it is difficult for the person to understand and know about these points over which the device needs to be kept, and measurement is to be taken. Constant participation of doctors and medical practitioners is therefore required.

Therefore, there is a need for an improved device that can provide assistance to a user in determining a target area over the body to keep the device and to draw accurate and fast results.

### SUMMARY

A reconfigurable wearable health monitoring device detachably attached to a smartwatch. For example, a reconfigurable wearable health monitoring device detachably attached to a smartwatch including a radio frequency (RF) sensing module, a semiconductor chip, an analog to digital converter (ADC), a processor, and an alignment feature. The radio frequency (RF) sensing module, having at least one transmitting antenna and at least one receiving antenna, configured to transmit radio frequency signals and receive a portion of responded radio frequency signals underneath or through the wrist of the user. The semiconductor chip configured to the convert the responded radio frequency signals into high-power radio frequency signals. The analog to digital converter (ADC) configured to receive the high-power radio frequency signals and to convert the high-power radio frequency signals into digital signals. The processor coupled with the semiconductor chip and configured to convert the digital signals into output information. The alignment feature configured with a rotating disc, an array, or a slider array to align the transmitting antenna and the receiving antenna over a target vein without moving the smartwatch.

In one example, a wearable health monitoring device for detachable attachment to a smartwatch. The wearable health monitoring device includes at least one antenna band, a base module and an alignment feature. The at least one antenna band includes a plurality of transmit antenna configured to transmit radio frequency (RF) detection signals into a skin surface of a user; and a plurality of receive antenna configured to receive responded RF detection signals that result from the RF detection signals transmitted into the user. The base module is configured for attachment to a bottom surface of the smartwatch. The base module includes a semiconductor chip configured to convert the responded RF detection signals into high-power RF signals; an analog to digital converter (ADC) configured to receive the high-power RF signals and to convert the high-power RF signals into digital signals; and a processor coupled with the semiconductor chip and configured to convert the digital signals into output information. The alignment feature assembly is for positioning antenna bands over a target vein without necessitating smartwatch movement. The alignment feature assembly includes a rotating disc, coupled to the base module via a central spindle having a central axis and a connector, the rotating disc rotatable about the central axis and having a bottom surface on which the at least one antenna band is located.

In another example, a wearable health monitoring device for detachable attachment to a smartwatch. The wearable health monitoring device includes at least one antenna band, a base module and an alignment feature. The at least one antenna band includes a plurality of transmit antenna configured to transmit radio frequency (RF) detection signals into a skin surface of a user; and a plurality of receive antenna configured to receive responded RF detection signals that result from the RF detection signals transmitted into the user. The base module is configured for attachment to a bottom surface of the smartwatch. The base module includes a semiconductor chip configured to convert the responded RF detection signals into high-power RF signals; an analog to digital converter (ADC) configured to receive the high-power RF signals and to convert the high-power RF signals into digital signals; and a processor coupled with the semiconductor chip and configured to convert the digital signals into output information. The alignment feature assembly is for positioning antenna bands over a target vein without necessitating smartwatch movement. The alignment feature assembly includes a slider coupled to the base module via one or more rails underneath the base module, the slider further coupled to the base module by a linking cable that enables the processor to communicate with the antenna bands, and the slider configured to slide across the base module; and a bottom slider surface in which the at least one antenna band is fabricated.

In another example, a wearable health monitoring device for detachable attachment to a smartwatch. The wearable health monitoring device includes at least one antenna band, a base module and an alignment feature. The at least one antenna band includes a plurality of transmit antenna configured to transmit radio frequency (RF) detection signals into a skin surface of a user; and a plurality of receive antenna configured to receive responded RF detection signals that result from the RF detection signals transmitted into the user. The base module is configured for attachment to a bottom surface of the smartwatch. The base module includes a semiconductor chip configured to convert the responded RF detection signals into high-power RF signals; an analog to digital converter (ADC) configured to receive the high-power RF signals and to convert the high-power RF signals into digital signals; and a processor coupled with the semiconductor chip and configured to convert the digital signals into output information. The alignment feature assembly is for positioning antenna bands over a target vein without necessitating smartwatch movement. The alignment feature assembly includes a panel, attached directly to the base module, including an array of antenna bands from the at least one antenna band on the bottom surface of the panel arranged in rows and columns.

In another example, a wearable health monitoring device for detachable attachment to a smartwatch. The wearable health monitoring device includes at least one antenna band, a base module and an alignment feature. The at least one antenna band includes a plurality of transmit antenna configured to transmit radio frequency (RF) detection signals into a skin surface of a user; and a plurality of receive antenna configured to receive responded RF detection signals that result from the RF detection signals transmitted into the user. The base module is configured for attachment to a bottom surface of the smartwatch. The base module includes a semiconductor chip configured to convert the responded RF detection signals into high-power RF signals; an analog to digital converter (ADC) configured to receive the high-power RF signals and to convert the high-power RF signals into digital signals; and a processor coupled with the semiconductor chip and configured to convert the digital signals into output information. The alignment feature assembly is for positioning antenna bands over a target vein without necessitating smartwatch movement. The alignment feature assembly includes at least one of (a) a rotating disc, coupled to the base module via a central spindle having a central axis and a connector, the rotating disc rotatable about the central axis and having a bottom surface on which the at least one antenna band is located; (b) a slider coupled to the base module via one or more rails underneath the base module, the slider further coupled to the base module by a linking cable that enables the processor to communicate with the antenna bands, and the slider configured to slide across the base module; and a bottom slider surface in which the at least one antenna band is fabricated; and (c) a panel, attached directly to the base module, including an array of antenna bands from the at least one antenna band on the bottom surface of the panel arranged in rows and columns.

In another example, a reconfigurable wearable health monitoring device detachably attached to a smartwatch to be worn by a user. The device includes a radio frequency (RF) sensing module having at least one transmitting antenna configured to transmit radio frequency signals underneath the skin surface of the user; at least one receiving antenna configured to receive reflected low-power radio frequency signals; and a semiconductor chip configured to convert the low power radio frequency signals into high power radio frequency signals. A processor is configured to convert the digital signals into output information. A wireless communication module coupled with the processor is configured to transmit the output information to the smartwatch to reflect the user's health parameters. An alignment feature is configured with a rotating disc, an array, or a slider array to align the transmitting antenna and receiving antenna over a target vein without moving the smartwatch.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. In some examples, one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a top view of a known smartwatch worn by a user;

FIGS. 2A-2C illustrate a conventional technique of aligning a smartwatch with respect to a target vein;

FIG. 3A illustrates a top view of a smartwatch coupled with a wearable health monitoring device, according to an embodiment;

FIG. 3B illustrates a cross-sectional view of the wearable health monitoring device, according to an embodiment;

FIG. 3C illustrates a top view of a rotating disc of the wearable health monitoring device with one or more transmitting and receiving antenna bands rotated over the skin surface of the user, according to an embodiment;

FIG. 3D illustrates a cross-sectional view of the smartwatch coupled with the wearable health monitoring device, according to another embodiment;

FIG. 3E illustrates a top view of the wearable health monitoring device and the smartwatch worn by the user, according to an embodiment;

FIG. 4A illustrates a top view of the smartwatch paired with the wearable health monitoring device, according to another embodiment;

FIG. 4B illustrates a cross-sectional view of the wearable health monitoring device of FIG. 4A;

FIG. 4C illustrates a transmitting and receiving antenna band of the wearable health monitoring device of FIG. 4B;

FIG. 4D illustrates an array of the transmitting and receiving antenna bands of FIG. 4C;

FIG. 5A illustrates a top view of the smartwatch paired with the wearable health monitoring device, according to another embodiment;

FIG. 5B illustrates a cross-sectional view of the wearable health monitoring device of FIG. 5A;

FIG. 5C illustrates a lateral movement of a slider of the wearable health monitoring device of FIGS. 5A-B with respect to a base module; and

FIG. 5D illustrates the slider's lateral movement with respect to the user's skin surface.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

FIG. 1 illustrates a top view of a known prior art wearable health monitoring device 100 coupled with a smartwatch 102 worn over a wrist 104 of a user.

The wearable health monitoring device 100 may be coupled with the smartwatch 102 and worn over the wrist 104 by the user via a watch strap 106. The wearable health monitoring device 100 may be configured to calculate the user's blood glucose level and other health parameters. In one embodiment, the wearable health monitoring device 100 and the smartwatch 102 may be specifically placed over a target vein 108 of the user's hand 110 to measure the glucose level. Further information on the construction and operation of the wearable health monitoring device 100 and the smartwatch 102 can be found in U.S. 2020/0187820 the entire contents of which are incorporated herein by reference.

In one embodiment, the wearable health monitoring device 100 may comprise a semiconductor chip (not shown) having a radio frequency sensing module (not shown), a memory unit (not shown), a processing unit (not shown), a communication module (not shown) and a battery (not shown). Further, the radio frequency sensing module may comprise at least one transmitting antenna (not shown) and at least one receiving antenna (not shown). In one alternative embodiment, the wearable health monitoring device 100 may be communicatively coupled to multiple devices, including a smart phone, a fitness band, a display unit, a touch screen, etc.

In one embodiment, the semiconductor chip may be configured to generate the radio frequency signals in a frequency range of 120-128 GHz. Alternatively, the semiconductor chip may comprise a phase-locked loop (PLL) (not shown), a band pass filter (BPF), and a frequency mixer (not shown) fabricated over the semiconductor chip. For instance, the semiconductor chip generates an analog signal at a frequency range of 9-11 MHz, and this 9-11 MHz is fed to the PLL, which generates an analog signal in the range of 3-5 GHz frequency. Further, the 3-5 GHZ signal is provided to the BPF, which filters the analog signal and passes a signal in the 3-5 GHz range to the mixer.

Further, the semiconductor chip may comprise a frequency synthesizer (not shown) and a frequency multiplier (not shown). The frequency synthesizer is configured to generate a variety of output frequencies as multiples of a single frequency. Further, the frequency multiplier is configured to generate an output frequency that is an odd or even multiple of its input frequency. Further, the semiconductor chip may comprise a frequency mixer (not shown) configured to create new frequencies with respect to applied frequencies.

For example, the frequency synthesizer uses the 9-11 MHz signal to produce a 16 GHz signal. The 16 GHz signal is fed to the frequency multiplier to generate a signal at 120 GHz by doubling the frequency. Further, the produced 3-5 GHz signals and the 120 GHz signals are mixed to generate a signal at 122-120 GHz depending on the frequency between the 3-5 GHz signal. Further, the signals are amplified in the 122-128 GHz range. These signals are transmitted under the user's skin surface by at least one transmitting antenna.

Further, the RF signals are reflected from the user and received by at least one receiving antenna in the form of electromagnetic energy, which may be further converted into electrical signals. For example, the electromagnetic energy in the 122-128 GHz frequency band is received by at least one receiving antenna and converted to a 122-128 GHz electrical signal. The semiconductor chip is configured to amplify the 122-128 GHz electrical signal into a 122-128 GHz frequency range to an amplified output signal. The amplified 122-128 GHz signal is mixed with the 128 GHz signal from the frequency multiplier with the received 122-128 GHz signal to generate a 3-5 GHz signal corresponding to the electromagnetic energy received at least one receiving antenna. The 3-5 GHz signal is mixed with the 3-5 GHz+2.5 MHz signal to generate a 2.5 MHz signal corresponding to the electromagnetic energy received by at least one receiving antenna. Further, the semiconductor chip uses the 2 GHz signal and mixes with the 2 GHz+2.5 MHz signals to generate a 2.5 MHz signal. Successively, the 2.5 MHz signal that corresponds to the electromagnetic energy is converted from an analog signal to a digital signal.

Further, the digital signals are received by the coupled processing unit to the semiconductor chip. Examples of implementations of the processing unit may include an X86-based processor, a Reduced Instruction Set Computing (RISC) processor, an Application-Specific Integrated Circuit (ASIC) processor, a Complex Instruction Set Computing (CISC) processor, and/or other processors. The processing unit is configured to convert the digital signal into digital data encoded with the health parameter information of the user. In one embodiment, the processing unit first filters the 2.5 MHz signal to remove the negative frequency spectrum and noise outside the desired bandwidth and converts the 2.5 MHz signal to digital data. Further, the semiconductor chip may comprise a decimation filter (not shown) configured to reduce the sampling frequency of the received signal. It can be noted that the decimation filter may be integrated with an analog-to-digital converter (ADC) within the semiconductor chip. The processing unit further decimated the digital data sampled at 9-11 MHz. It may be noted that the digital data is decimated to reduce the amount of data by selectively discarding a portion of sampled data to uncover meaningful information from the digital data.

The output of the decimation filter is the digital data representative of the electromagnetic energy received at the corresponding antenna. Alternatively, signal processing techniques may be used to achieve beamforming, Doppler effect processing, and/or leakage mitigation in order to separate a desired signal from other undesirable signals. The digital signal processing of incoming signals may use Kalman filters to smooth out noisy data. In one aspect, the digital signal processing of received signals may include digitally merging receive chains. In another aspect, multiple digital signal processing techniques may be utilized to achieve beamforming, Doppler effect processing, and range. Digital signal processing can be accomplished in a digital signal processor (DSP).

Further, millimeter-range radio frequency signals may be delivered beneath the skin to highlight anatomical characteristics. Blood flowing via veins such as the basilic and cephalic veins moves relative to other anatomical characteristics in the region surrounding the wrist 104. Thus, the processing unit employs the Doppler effect theory and associated signal processing to filter for signals corresponding to blood flowing inside veins relative to other signals corresponding to stationary objects. It can be noted that the stationary objects may correspond to bone and fibrous tissue such as muscle and tendons. The signals that correspond to the flowing blood may be recognized and isolated. The isolated signals may then be utilized to calculate a health metric, such as blood glucose levels.

In one embodiment, the Doppler effect theory may be applied for processing received signals to separate the signals corresponding to the flowing blood moving relative to the transmitting and receiving antennas from the signals corresponding to stationary objects. Although the approaches discussed above focus on monitoring the blood glucose level, the disclosed techniques may also be relevant to monitor other health metrics such as blood pressure and heart rate.

In an embodiment, the digital data processed by the processing unit is further saved within the memory unit. The memory unit may include suitable logic, circuitry, and/or interfaces that may be configured to store a machine code and/or a computer program with at least one code section executable by the processor unit. Examples of implementation of the memory may include, but are not limited to, Random Access Memory (RAM), Read Only Memory (ROM), Hard Disk Drive (HDD), and/or a Secure Digital (SD) card. In an embodiment, the digital information processed by the processing unit is saved within the memory unit in the form of a database.

Further, the database may be segregated into a raw data sample and derived data fetched from the user. In one embodiment, the raw data sample may correspond to standard data or a threshold limit for performing a specific health parameter. The derived data may correspond to data calculated or evaluated from a sample in one embodiment. The raw data sample may be used as a training data set as well as a reference point for the wearable health monitoring device 100 to determine the accurate health results of the user. It may be noted that the raw data sample may include data sets of different parameters to allow the wearable health monitoring device 100 to fetch the derived data at different conditions. In one embodiment, the raw data samples include data sets specific to gender, age, specific to weather conditions, specific to regions, etc.

In an embodiment, the wearable health monitoring device 100 may also be integrated with the communication module. The communication module may be configured to transfer the derived data stored within the database to the smartwatch 102 for the user. Further, the communication module may provide a medium through which the wearable health monitoring device 100 may communicate with a cloud network in the network environment or with each other. Such communication may facilitate the user to securely save the data in a cloud database and enable access to the derived data from multiple locations and devices.

Examples of the communication network may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). Various devices may be configured to connect the communication network to the cloud via various wired and wireless communication protocols. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols. In an embodiment, the wearable health monitoring device 100 may also be integrated with the battery that may be configured to power all the elements of the wearable health monitoring device 100.

In one embodiment, the location of the transmitting and the receiving antennas may be aligned along the target vein 108 to measure the accurate glucose level of the user. In order to determine accurate results, it is desirable to have the transmitting and the receiving antennas in contact with or close to the skin (e.g., within 3-5 mm) aligned with the target vein 108. The transmitting and the receiving antennas may be positioned relative to the target vein 108 such that the target vein 108 is positioned just below a center line 112 between an array of the transmitting and the receiving antennas.

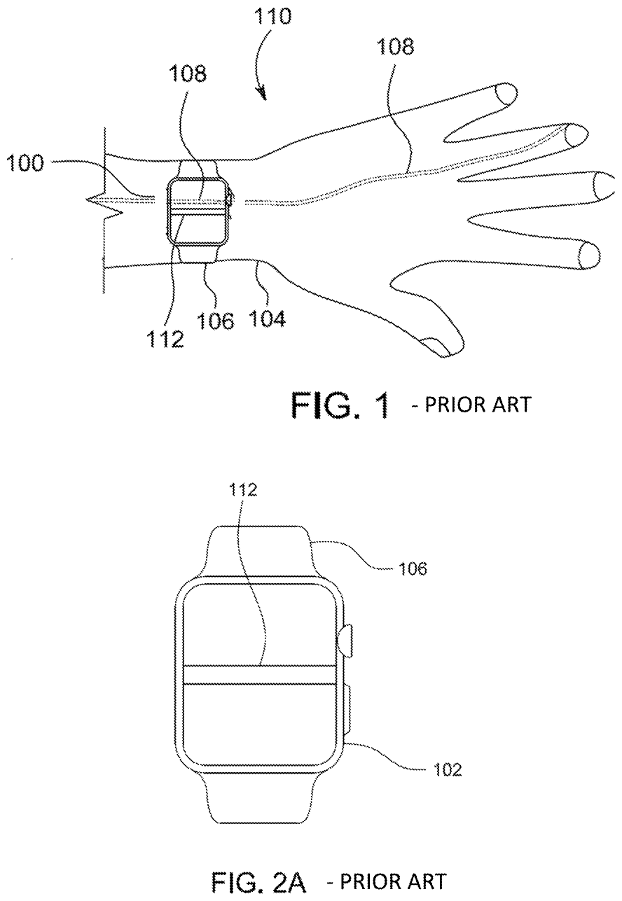

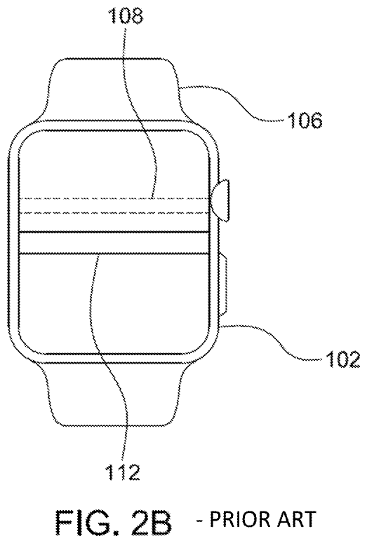

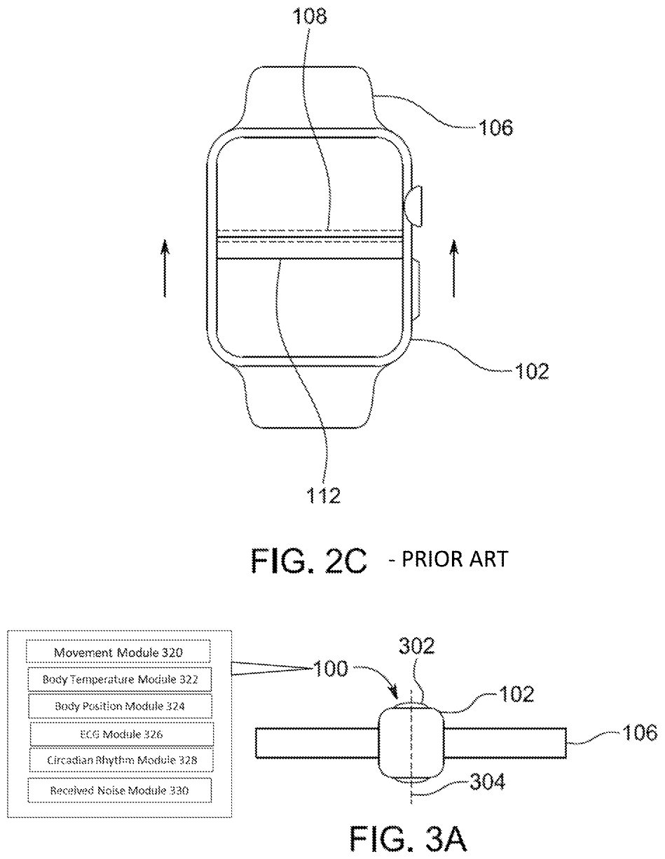

FIGS. 2A-2C illustrate the center line 112 displayed on the smartwatch 102 from the prior art with respect to the target vein 108, according to an embodiment.

In one embodiment, the center line 112 may be aligned just above the target vein 108. In another embodiment, the center line 112 marked over the smartwatch 102 may be misaligned from the target vein 108. In another embodiment, the center line 112 marked over the smartwatch 102 may be slightly misaligned from the target vein 108.

It can be noted that a measure of alignment entails measuring the distance of the target vein 108 from the center line 112. In one embodiment, the center line 112 may be defined as an imaginary line between the transmitting and receiving antennas. In another embodiment, the distance may be calculated as a lateral distance of the target vein 108 from the center line 112 at more than one site. In another embodiment, the distance may be calculated as the average distance of the target vein 108 from the center line 112. It may be noted that other techniques may be used for obtaining alignment between the target vein 108 and the center line 112. In one embodiment, an antenna array may be aligned with the target vein 108 when a centerline of a two-dimensional array of receive antennas is within a threshold distance to the target vein 108. The threshold distance between the target vein 108 and the center line 112 may range from 1 millimeter (mm) to 3 mm.

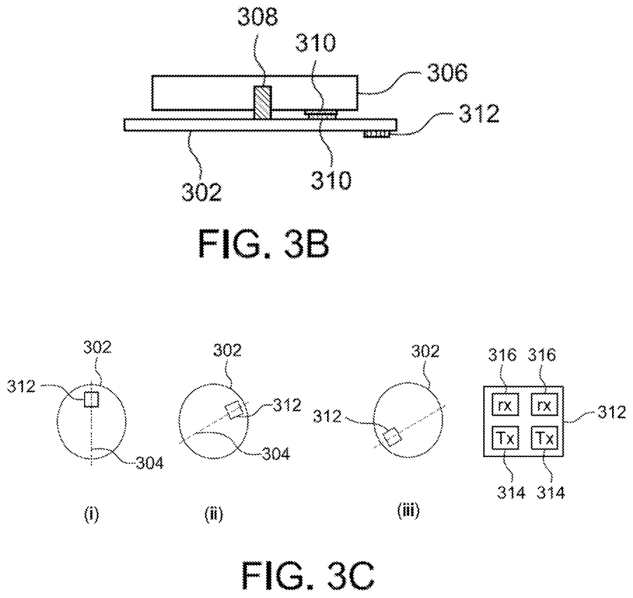

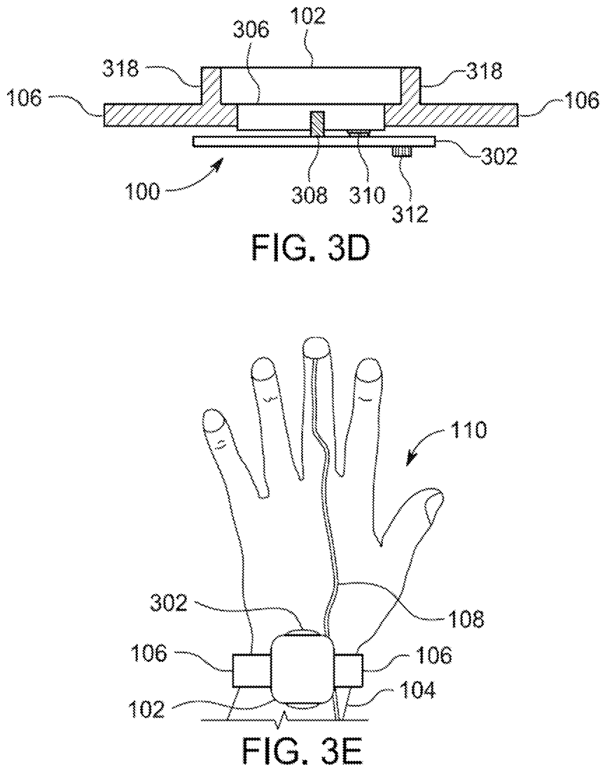

FIG. 3A illustrates a top view of the smartwatch 102 coupled with the wearable health monitoring device 100, according to an embodiment. FIG. 3A may be described in conjunction with FIGS. 3B-3E. This smartwatch 102 is well known in the prior art that may contain a Bluetooth or other communications device. The smartwatch 102 will sync with the wearable health monitoring device 100, which has the systems to be able to send and receive Activated RF Ranges (500 MHZ to 300 GHZ) and to be able to process the received signals to a level for post processing in the smartwatch 102. The smartwatch 102 has an application that can use the received and communicated signals to convert the received signals to a glucose level or other data and be able to display it to the user using the display on smartwatch 102.

The wearable health monitoring device 100 may comprise a rotating disc 302 and a central axis line 304. The rotating disc 302 may be configured to be rotated in a clockwise and/or counterclockwise direction by the user (when viewing the disc 302 from above like in FIG. 3C). In another embodiment the rotating disc can be rotated automatically using a small motor drive or other drive mechanism. The movement of the rotating disc 302 may be shown with reference to the central axis line 304. Further, the wearable health monitoring device 100 may comprise a base module 306, a central spindle 308, a connector 310, and one or more transmitting and receiving antenna bands 312 (as shown in FIG. 3B). By module we mean either executable software or hardware or combinations of both. Base module 306 may contain a memory, a processor, a power source (battery), a comms (Bluetooth or Wi-Fi), a connector to the antenna bands 312, a signal processing unit, RF amplifier and RF transmitters, RF filters signal conditions, and a central spindle 308. Base module performs the function of sending signals out of the TX antennas, receiving RF signals from the RX antennas, then processing the signals using the RF amplifiers, RF filters, signal processing unit using processing and memory, processing the signals to send to the smartwatch 102 through the comms of the base module 306 and the smartwatch 102.

The base module 306 may be coupled to the rotating disc 302 via the connector 310 and the central spindle 308. The rotating disc 302 may rotate about an axis of the central spindle 308. In one embodiment, the axis of the rotating disc 302 may correspond to the central axis line 304, displayed over the smartwatch 102. In one embodiment, the base module 306 may be sandwiched between the bottom surface (not shown) of the smartwatch 102 and the top surface (not shown) of the rotating disc 302. Axis line 304 is orthogonal to central spindle 308. Central spindle 308 allows the disc 302 to rotate clockwise or counterclockwise.

Further, the base module 306 may adhere underneath the smartwatch 102. In one embodiment, the base module 306 may be affixed to the base of the smartwatch 102 using fastening means (such as adhesive, suction cup, or magnetic connector). The fastening means may also include a press-fit mechanism, hook and loop fasteners, chemical fixtures, etc. In an alternative embodiment, the base module 306 may be detachably coupled to the bottom surface of the smartwatch 102 via a clipping component, such as a mechanical structure connected to the smartwatch 102 and allowing base module 306 to be inserted into the mechanical structure (not shown) or a fastening component (not shown). In one embodiment, the base module 306 may comprise the radio frequency sensing module having at least one transmitting antenna and at least one receiving antenna. Base module 306 main contain a memory, a processor, a power source (battery), a comms (Bluetooth or Wi-Fi), a connector to the antenna bands 312, a signal processing unit, RF amplifier and RF transmitters, RF filters signal conditions, and central spindle 308. Base module performs the function of sending signals out the TX antennas, receiving RF signals from the RX antennas, then processing the signals using the RF amplifiers, RF filters, signal processing unit using processing and memory, processing the signals to send to the smartwatch 102 through the comms of the base module 306 and the smartwatch 102.

It can be noted that the communication module may be configured to allow transmitting information from the base module 306 to the smartwatch 102 or vice versa.

Further, the wearable health monitoring device 100 may comprise the central spindle 308. The central spindle 308 may be interconnected between the base module 306 and the rotating disc 302 such that the rotating disc 302 is rotatable in a clockwise and/or counterclockwise direction. Further, the wearable health monitoring device 100 may comprise the connector 310. In one embodiment, the connector 310 may be configured to enable electrical connection between the transmitting and receiving antenna band 312 and the processing unit integrated within the base module 306. Further, the transmitting and receiving antenna band 312 may adhere underneath the rotating disc 302 such that, once the smartwatch 102 along with the wearable health monitoring device 100 is worn by the user over the wrist 104, the transmitting and receiving antenna band 312 may be placed in close contact with the skin of the user.

FIG. 3C illustrates a top view of the rotating disc 302 with transmitting and receiving antenna band 312 rotated at multiple angular positions about an axis of the central spindle 308, according to an embodiment. Disc 302 rotates about an axis of the spindle.

In one embodiment, the transmitting and receiving antenna band 312 may include one or more transmitting antennas 314 and one or more receiving antennas 316 placed in a parallel configuration with each other. In one embodiment, the transmitting and receiving antenna band 312 may rotate in a clockwise and counterclockwise direction with the rotating disc 302.

In one exemplary embodiment, the transmitting and receiving antenna band 312 is positioned by the angular movement of the rotating disc 302 to align the transmitting and receiving antenna band 312 with the target vein 108. At position (i), the transmitting and receiving antenna band 312 is in alignment with the central axis line 304. As the rotating disc 302 is turned clockwise (or counterclockwise), the transmitting and receiving antenna band 312 reaches position (ii). In this position, the transmitting and receiving antenna band 312 have moved to a second position so that receiving antennas are in a different place on the users wrist. Further, as the rotating disc 302 is further rotated by the user in a clockwise (or counterclockwise) direction, the transmitting and receiving antenna band 312 reaches a position (iii).

FIG. 3D illustrates a cross-sectional view of the smartwatch 102 coupled with the wearable health monitoring device 100, according to an embodiment.

The wearable health monitoring device 100 may be coupled to the watch strap 106 of the smartwatch 102 using one or more connecting mounts 318. The rotating disc 302 may be rotated with a space between the rotating disc 302 and the base module 306. The width of the space may be equal to the width of the connector 310. In one exemplary embodiment, the transmitting and receiving antenna band 312 may be flush with the surface of the rotating disc 302. In another exemplary embodiment, the transmitting and receiving antenna band 312 may be layered with a glass surface (not shown). It can be noted that a smooth bottom surface of the rotating disc 302 may prevent rough and direct interaction of the transmitting and receiving antenna band 312 with the skin of the user and enable free movement of the rotating disc 302.

FIG. 3E illustrates a top view of the wearable health monitoring device 100 worn over the wrist 104 of the user, according to an embodiment.

The rotating disc 302 may be rotated to cover multiple veins passing through the wrist 104 of the user. As discussed earlier, as the rotating disc 302 is turned by the user in a clockwise (or counterclockwise) direction, the transmitting and receiving antenna band 312 reaches position (ii), as shown in FIG. 3C. Such rotation of the rotating disc 302 may allow the user to align the transmitting and receiving antenna band 312 with at least one vein. Further, as the rotating disc 302 is rotated by the user in a clockwise (or counterclockwise) direction, the transmitting and receiving antenna band 312 reaches a position (iii), as shown in FIG. 3C, to cover other veins. Therefore, the rotation of the rotating disc 302 may enhance the versatility of the wearable health monitoring device 100.

In one embodiment, the receiving and transmitting antenna band 312 may be configured to continuously emit and then receive radio waves, as the rotating disc 302 is turned by the user in the clockwise and/or counterclockwise direction. Once the receiving and transmitting antenna band 312 gets perfectly aligned over the target vein 108, the smartwatch 102 receives constant notification as to the antennas antenna bands 312 position relative to the vein. For instance, if the RX antennas are not over the vein, the signals received by the RX antennas and processes using base module 306, may provide a weak signal. However, when the RX antennas 312 are directly over a vein, the signals received by the RX antennas and processes would be much stronger. The smartwatch 102 receives all the signals through the comms interface and displays them on the smartwatch 102 display. In one embodiment, the notification may be a visual or could be voice message, a specific vibration, or other human perceptible notification.

For example, Alex wears the smartwatch 102 with the wearable health monitoring device 100 over his left wrist and aligns the transmitting and the receiving antenna band 312 with the target vein 108. The transmitting and receiving antenna band 312 continuously transmits and receives radio frequency signals of range 120-128 MHz into and from the wrist 104. As Alex rotates the rotating disc 302 at 25 degrees north-west with respect to the smartwatch 102, the smartwatch 102 sends a notification that target vein 108 is detected and to hold the wearable health monitoring device 100 in the aligned position. The received signals at this position are converted and processed to generate output information. This output information corresponds blood glucose level of 110 mg/dL; Heart rate of 72 BPM; Blood Pressure of 110/70; and SpO2 of 97%.

The health monitoring device 100 may include a movement module 320 that includes at least one sensor from the group of an accelerometer, a gyroscope, an inertial movement sensor, or other similar sensor. The movement module 320 may have its own processor or utilize the semiconductor chip to calculate movement of the user. Motion from the user will change the blood volume in a given portion of their body, and flow rate of blood in their circulatory system. This may cause noise, artifacts, or other errors in the real-time signals received by the receiving antenna. The movement module 320 may compare the calculated motion to a threshold motion stored in memory unit. For example, the motion threshold could be movement of more than two centimeters in a one second period. The motion threshold could be near zero to ensure the user is stationary when measuring to ensure the least noise in the RF signal data. When calculated motion levels exceeds the threshold the movement module 320 may flag the RF signals collected at the time stamp corresponding to the motion as potentially being inaccurate. In some embodiments, the movement module 320 may compare RF signal data to motion data over time to improve the accuracy of the movement threshold. The movement module may alert the user, such as with an audible beep or warning, or a text message or alert to a connected mobile device. The alert would signal to the user that they are moving too much to get an accurate measurement.

The health monitoring device 100 may include a body temperature module 322 that includes at least one sensor from the group of a thermometer, a platinum resistance thermometer (PRT), a thermistor, a thermocouple, or other temperature sensor. The body temperature module 322 may have its own processor or utilize the semiconductor chip to calculate the temperature of the user or the user's environment. The user's body temperature, the environmental temperature, and the difference between the two will change the blood volume in a given part of their body, and flow rate of blood in their circulatory system. Variations in temperature from normal body temperature or room temperature may cause noise, artifacts, or other errors in the real-time signals received by the receive antennas. The body temperature module 322 may compare the measured temperature to a threshold temperature stored in memory unit. For example, the environmental temperature threshold may be set at zero degrees Celsius because low temperatures can cause a temporary narrowing of blood vessels which may increase the user's blood pressure. When the measured temperature exceeds the threshold the body temperature module 322 may flag the RF signals collected at the time stamp corresponding to the temperature as potentially being inaccurate. In some embodiments, the body temperature module 322 may compare RF signal data to temperature data over time to improve the accuracy of the temperature threshold. The body temperature module 322 may alert the user, such as with an audible beep or warning, or a text message or alert to a connected mobile device. The alert would signal to the user that their body temperature, or the environmental temperature is not conducive to getting an accurate measurement.

The health monitoring device 100 may include a body position module 324 that includes at least one sensor from the group of an accelerometer, a gyroscope, an inertial movement sensor, or other similar sensor. The body position module 324 may have its own processor or utilize the semiconductor chip to estimate the position of the user. The user's body position may change the blood volume in a given part of their body, and flow rate of blood in their circulatory system. This may cause noise, artifacts, or other errors in the real-time signals received by the receive antennas. The body position module 324 may compare the estimated position to a body position threshold stored in memory unit. For example, the smartwatch 102 may be on the user's wrist and the body position threshold may be based on the relative position of the user's hand to their heart. When a user's hand is lower than their heart, their blood pressure will increase, with this effect being more pronounced the longer the position is maintained. Conversely, the higher above a user's holds their arm above their heart, the blood pressure in their hand will be lower. The body position threshold may include some minimum amount of time the estimated body position occurs. When the estimated position exceeds the threshold the body position module may flag the RF signals collected at the time stamp corresponding to the body position as potentially being inaccurate. In some embodiments, the body position module 324 may compare RF signal data to motion data over time to improve the accuracy of the body position threshold. The body position data may also be used to estimate variations is parameters such as blood pressure that correspond to the body position data so as to improve the accuracy of the measurements taken when the user in in that position. The body position module 324 may alert the user, such as with an audible beep or warning, or a text message or alert to a connected mobile device. The alert would signal to the user that their body position is not conducive to getting an accurate measurement.

The health monitoring device 100 may include an ECG module 326 that includes at least one electrocardiogram sensor. The ECG module 326 may have its own processor or utilize the semiconductor chip to record the electrical signals that correspond with the user's heartbeat. The user's heartbeat will impact blood flow. Measuring the ECG data may allow the received RF data to be associated with peak and minimum cardiac output so as to create a pulse wave form allowing for the estimation of blood volume at a given point in the wave of ECG data. Variations in blood volume may cause noise, artifacts, or other errors in the real-time signals received by the receive antennas. The ECG module 326 may compare the measured cardiac data to a threshold stored in memory unit. For example, the threshold may be a pulse above a 160 bpm, as the increased blood flow volume may cause too much noise in the received RF signal data to generate an accurate measure of blood glucose. When the ECG data exceeds the threshold the ECG module 326 may flag the RF signals collected at the time stamp corresponding to the ECG data as potentially being inaccurate. In some embodiments, the ECG module 326 may compare RF signal data to ECG data over time to improve the accuracy of the ECG data threshold or to improve the measurement of glucose at a given point in the cycle between peak and minimum cardiac output. The ECG module 326 may alert the user, such as with an audible beep or warning, or a text message or alert to a connected mobile device. The alert would signal to the user that their heart rate is not conducive to getting an accurate measurement or requires additional medical intervention.

The health monitoring device 100 may include a circadian rhythm module 328 that includes at least one sensor measuring actigraphy, wrist temperature, light exposure, and heart rate. The circadian rhythm module 328 may have its own processor or utilize the semiconductor chip to calculate the user's circadian health. Blood pressure follows a circadian rhythm in that it increases on waking in the morning and decreases during sleeping at night. People with poor circadian health will often have higher blood pressure. These variations in blood pressure can noise, artifacts, or other errors or inaccuracies in the real-time signals received by the receive antennas. The circadian rhythm module 328 may compare the circadian data to a threshold stored in memory unit. For example, the threshold may be set as less than 6 hours of sleep in the last 24 hours. When the observed circadian health data exceeds the threshold the circadian rhythm module may flag the RF signals collected at the time stamp corresponding to circadian health as potentially being inaccurate, or as needing an adjustment to account for the expected increase in the user's blood pressure. In some embodiments, the circadian rhythm module 328 may compare RF signal data to sleep data over time to improve the accuracy of the circadian rhythm thresholds. The circadian rhythm module 328 may alert the user, such as with an audible beep or warning, or a text message or alert to a connected mobile device. The alert would signal to the user that their recent sleep patterns are not conducive to getting an accurate measurement.

The health monitoring device 100 may include a received noise module 330 that includes at least one sensor measuring background signals such as RF signals, Wi-Fi, and other electromagnetic signals that could interfere with the signals received by the receive antennas. The received noise module 330 may have its own processor or utilize the semiconductor chip to calculate the level of background noise being received. Background noise may interfere with or cause noise, artifacts, or other errors or inaccuracies in the real-time signals received by the receive antennas. The received noise module 330 may compare the level and type of background noise to a threshold stored in memory unit. The threshold may be in terms of field strength (volts per meter, and ampere per meter) or power density (watts per square meter). For example, the threshold may be RF radiation at greater than 300 μW/m2. When the background noise data exceeds the threshold the received noise module may flag the RF signals collected at the time stamp corresponding to background noise levels as potentially being inaccurate. In some embodiments, the received noise module 330 may compare RF signal data to background noise over time to improve the accuracy of the noise thresholds. The received noise module 330 may alert the user, such as with an audible beep or warning, or a text message or alert to a connected mobile device. The alert would signal to the user that the current level of background noise is not conducive to getting an accurate measurement.

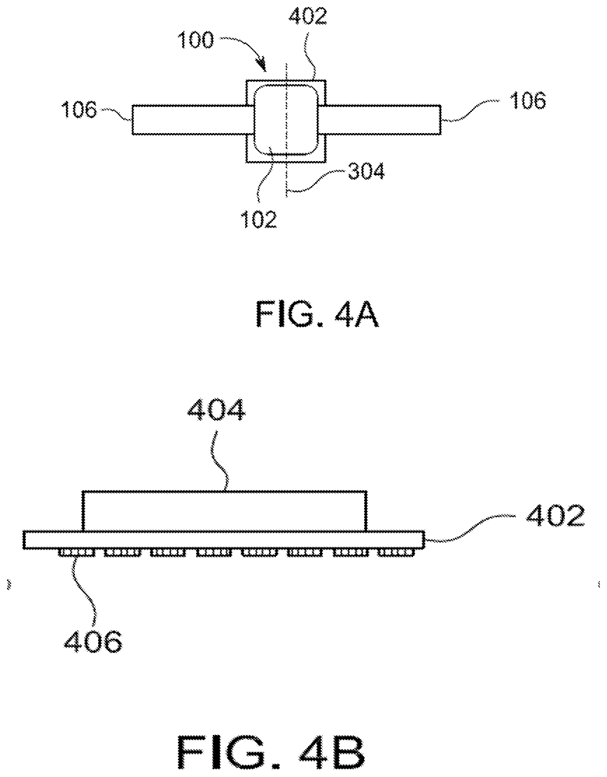

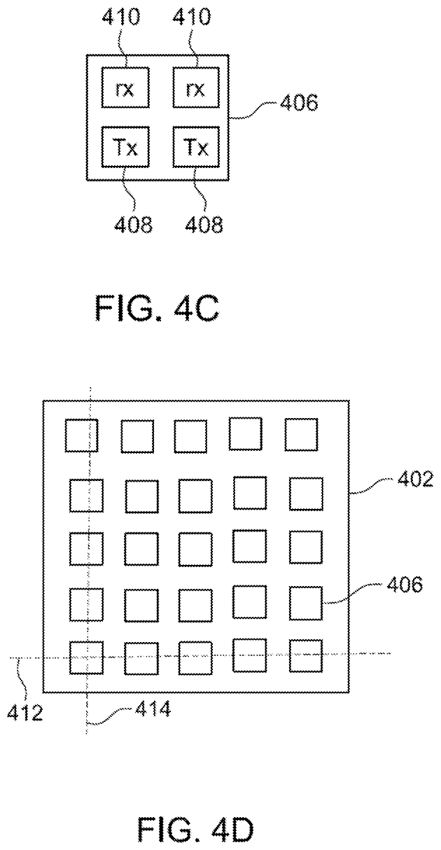

FIG. 4A illustrates a top view of the smartwatch 102 paired with the wearable health monitoring device 100, according to an alternate embodiment. FIG. 4A may be described in conjunction with FIGS. 4B-4D.

The smartwatch 102 may be detachably coupled with the wearable health monitoring device 100. The wearable health monitoring device 100 may comprise a panel 402. Further, the panel 402 may be attached to a base module 404. In one embodiment, the base module 404 may comprise the semiconductor chip with TX and RX antennas as well as the radio frequency sensing module, the memory unit, the processing unit, the communication module, and the battery, as discussed above. Further, the panel 402 may comprise a plurality of transmitting and receiving antenna bands 406. Further, each of the plurality of transmitting and receiving antenna bands 406 may comprise at least two transmitting antennas 408 and at least two receiving antennas 410, as shown in FIG. 4C. Further base module 404 main contain a memory, a processor, a power source (battery), a comms (Bluetooth or Wi-Fi), a connector to the RX TX antennas (406), a signal processing unit, RF amplifier and RF transmitters, RF filters signal conditions, and a central spindle 308. Base module performs the function of sending signals out the TX antennas, receiving RF signals from the RX antennas, then processing the signals using the RF amplifiers, RF filters, signal processing unit using processing and memory, processing the signals to send to the smartwatch 102 through the comms of the base module 404 and the smartwatch 102.

In one embodiment, the panel 402 may comprise an array of transmitting and receiving antenna bands 406 in rows 412 and columns 414, as shown in FIG. 4D. The array of transmitting and receiving antenna bands 406 in rows 412 and columns 414 may be configured to cover the maximum area over the surface of the wrist 104 of the user with at least one of the plurality of transmitting and receiving antenna bands 406. In one embodiment, the processing unit embedded within the wearable health monitoring device 100 may be configured with a method to sequentially activate and deactivate each of the plurality of transmitting and receiving antenna bands 406. Such activation and deactivation of the plurality of transmitting and receiving antenna bands 406 may allow to pick up of radio signals back from at least one target vein.

In one exemplary embodiment, Alex wears the smartwatch 102 and the wearable health monitoring device 100 over his left wrist and aligns the plurality of transmitting and receiving antenna bands 406 of the wearable health monitoring device 100 with the target vein 108. The plurality of transmitting and receiving antenna bands 406 may either continuously or sequentially transmit and receive radio frequency signals of range 120-128 GHz into and from the wrist 104, as programmed with the processing unit. For example, one transmitting and receiving antenna band 406 with coordinate (1,1), which corresponds to a transmitting and receiving antenna band 406 at the top left of the array, gets activated first to emit and receive radio frequency signals of range 120-128 GHz. In case the target vein 108 is not detected, the processing unit activates a next transmitting and receiving antenna band 406 of the plurality of transmitting and receiving antenna bands 406 with coordinate (1,2).

Successively, as the transmitting and the receiving antenna band 406 with coordinate (1,2) gets activated, the processing unit receives a reflected radio signal from the target vein 108 to confirm the presence of the target vein 108. Further, the communication module of the wearable health monitoring device 100 sends the notification to the smartwatch 102 that target vein 108 is detected. The received signals at this location are converted and processed to generate output information. This output information corresponds to a blood glucose level of 111 mg/dL; a heart rate of 76 BPM; a blood pressure of 110/70; and SpO2 of 96.5%.

In an alternative embodiment, the processing unit may be configured to individually activate a transmitting antenna of one transmitting and receiving antenna band 406 and a receiving antenna of another transmitting and receiving antenna band 406. Such activation of the processing unit may facilitate the plurality of transmitting and receiving antenna bands 406 to receive better-reflected radio signals from the target vein 108. In one example, Alex wears the smartwatch 102 and the wearable health monitoring device 100 over his left wrist and aligns the plurality of transmitting and receiving antenna bands 406 with the target vein 108. The transmitting antenna of one of the transmitting and receiving antenna bands 406 is activated, and the receiving antenna of the other transmitting and receiving antenna band 406 is activated simultaneously to transmit and receive radio frequency signals of range 120-128 GHz into and from the left wrist.

In another example, a transmitting antenna of the one transmitting and receiving antenna band 406 with coordinates (2,3) gets activated first, followed by a receiving antenna of the another transmitting and receiving antenna band 406 with coordinates (4,4), to emit and receive radio frequency signals of range 120-128 GHz. Further, the smartwatch 102 receives a notification that target vein 108 is detected. The received signals at this location are converted and processed to generate output information. This output information corresponds to a blood glucose level of 112 mg/dL; a heart rate of 77 BPM; a blood pressure of 114/69; and SpO2 of 97.5%.

In an alternative embodiment, the processing unit may be configured to activate the transmitting antennas and the receiving antennas of pre-configured groups of the plurality of transmitting and receiving antenna bands 406. Such configuration and grouping of the transmitting and receiving antennas may enable the wearable health monitoring device 100 to expand the range for identification of the target vein 108 and thereby provide highly accurate reflected radio signals.

For example, Alex wears the smartwatch 102 and the wearable health monitoring device 100 over his left wrist and aligns the plurality of transmitting and the receiving antenna bands 406 with the target vein 108. The transmitting antennas and the receiving antennas of two different groups of the plurality of transmitting and receiving antenna bands 406 are activated to transmit and receive radio frequency signals of range 120-128 MHz into and from the left wrist.

In one exemplary embodiment, a transmitting antennas group of the plurality of transmitting and receiving antenna bands 406 with coordinates (2,3: 3,3: 4,3) are activated first, followed by a receiving antennas group of the plurality of transmitting and receiving antenna bands 406 with coordinates (3,4: 4,4: 5,4), to receive radio waves. Further, the smartwatch 102 receives a notification that target vein 108 is detected. The received signals at this location are converted and processed to generate output information. This output information corresponds to a blood glucose level of 113 mg/dL; Heart rate of 75 BPM; Blood Pressure of 109/72; and SpO2 of 98%.

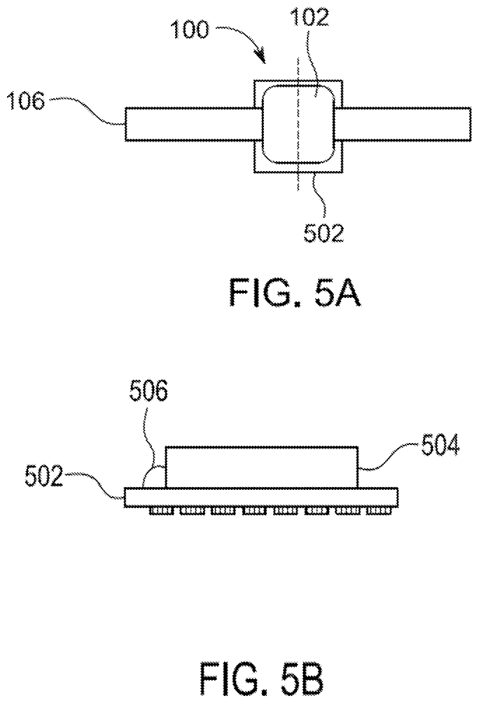

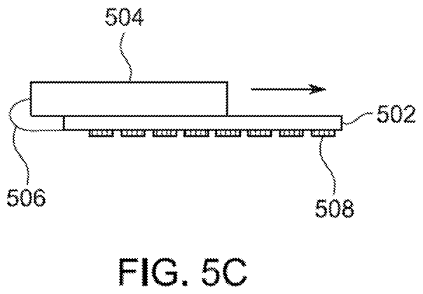

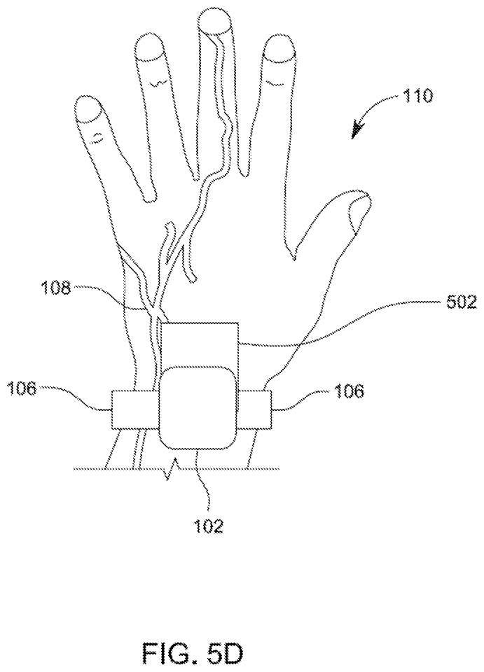

FIG. 5A illustrates a top view of the smartwatch 102 paired with the wearable health monitoring device 100, according to a second alternate embodiment. FIG. 5A may be described in conjunction with FIGS. 5B-5D.

The wearable health monitoring device 100 may comprise a slider 502, a base module 504, a linking cable 506, and a plurality of transmitting and receiving antenna bands 508. The base module 504 may have the same configuration as described in FIG. 3A. The slider 502 may be coupled underneath the base module 504 in a sliding configuration. Further, the base module 504 may be coupled to the slider 502 via the linking cable 506. The slider 502 may be configured to slide left or right (for example, when viewing FIG. 5B) across the length of the base module 504.

Further, the plurality of transmitting and the receiving antenna bands 508 may be fabricated underneath the slider 502. The plurality of transmitting and the receiving antenna bands such as 508 may comprise one or more transmit antenna and receive antenna bands. In one embodiment, each of the one or more transmit antenna and receive antenna bands 508 may be activated individually or in combination by the processing unit. The linking cable 506 may be configured to allow the processing unit to communicate with the plurality of transmitting and receiving antenna bands 508 through electrical signals. The sliding configuration may be facilitated by the flexible linking cable 506, which provides free movement to the slider 502 during the sliding movement. In one embodiment, the linking cable 506 may be made from a material selected from a group of materials such as, but not limited to, rubber, stretching polymer, polyethylene (PE), and steel. In one embodiment, the slider 502 may be configured with a sliding rail (not shown) that may enable free movement of the slider 502 with respect to the bottom surface of the base module 504. The sliding movement of the slider 502 may enable the plurality of transmitting and the receiving antenna bands 508 to have maximum area covered over the wrist 104 of the user, as shown in FIG. 5D. In one embodiment, the slider 502 may have the sliding movement towards the fingers or arms or towards one side of the wrist or the other. with respect to the wrist 104. The sliding movement of the slider 502 may enhance the detection of the target vein 108.

In one example, Alex wears the smartwatch 102 and the wearable health monitoring device 100 over his left wrist and starts aligning the plurality of transmitting and receiving antenna bands 508 with respect to the target vein 108. Each transmit antenna and receive antenna of the plurality of transmitting and receiving antenna bands 508 may either continuously or sequentially transmit and receive radio frequency signals of range 120-128 MHz underneath the wrist 104, as programmed with the processing unit. In another example, one transmit and receive antenna band 508 of the plurality of transmitting and receiving antenna bands 508 with coordinate (1, 1) is activated first to emit and receive radio waves. If the one transmit and receive antenna band 508 does not detect the presence of the target vein 108, the processing unit sends a notification to the smartwatch 102 suggesting to the user to slide the slider 502 towards the right side.

Further, after sliding the slider 502, the processing unit may repeat the process by activating another transmit and receive antenna band 508 with coordinates (1, 2) of the plurality of transmitting and receiving antenna bands 508. The processing unit receives a reflect radio signal from the target vein 108 to confirm the presence of the target vein 108. Further, the smartwatch 102 receives a notification that target vein 108 is detected. The received signals at this location are converted and processed to generate output information. This output information corresponds to a blood glucose level of 110 mg/dL; Heart rate of 74 BPM; Blood Pressure of 111/70; and SpO2 of 96.5%.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the subject disclosure as disclosed above.

What is claimed is:

1. A wearable health monitoring device for detachable attachment to a smartwatch, comprising:
at least one antenna band, including:
a plurality of transmit antenna configured to transmit radio frequency (RF) detection signals into a skin surface of a user; and
a plurality of receive antenna configured to receive responded RF detection signals that result from the RF detection signals transmitted into the user;
a base module configured for attachment to a bottom surface of the smartwatch, including:
a semiconductor chip configured to convert the responded RF detection signals into high-power RF signals;
an analog to digital converter (ADC) configured to receive the high-power RF signals and to convert the high-power RF signals into digital signals; and
a processor coupled with the semiconductor chip and configured to convert the digital signals into output information; and
an alignment feature assembly for positioning antenna bands over a target vein without necessitating smartwatch movement, comprising:
a rotating disc, coupled to the base module via a central spindle having a central axis and a connector, the rotating disc rotatable about the central axis and having a bottom surface on which the at least one antenna band is located.

2. The wearable health monitoring device of claim 1, wherein the alignment feature assembly includes a motor drive that automatically rotates the rotating disc.

3. The wearable health monitoring device of claim 1, wherein the plurality of transmit antenna and the plurality of receive antenna are placed in parallel configuration with each other.

4. The wearable health monitoring device of claim 1, wherein the at least one antenna band is flush with the bottom surface of the rotating disc.

5. The wearable health monitoring device of claim 1, wherein when at least one antenna band is well-aligned over the target vein, the smartwatch receives constant notifications including stronger signals the more directly the antenna bands are over the target vein; wherein notifications include one or more of: visual alerts, voice messages, or specific vibrations.

6. The wearable health monitoring device of claim 1, wherein the antenna bands continuously or sequentially transmit and receive RF detection signals as programmed with the processor.

7. The wearable health monitoring device of claim 1, wherein the antenna bands are configured to individually activate a transmit antenna of one antenna band and a receive antenna of another antenna band

8. A wearable health monitoring device for detachable attachment to a smartwatch, comprising:
at least one antenna band, including:
a plurality of transmit antenna configured to transmit radio frequency (RF) detection signals into a skin surface of a user; and
a plurality of receive antenna configured to receive responded RF detection signals that result from the RF detection signals transmitted into the user;
a base module configured for attachment to a bottom surface of the smartwatch, including:
a semiconductor chip configured to convert the responded RF detection signals into high-power RF signals;
an analog to digital converter (ADC) configured to receive the high-power RF signals and to convert the high-power RF signals into digital signals; and
a processor coupled with the semiconductor chip and configured to convert the digital signals into output information; and
an alignment feature assembly for positioning antenna bands over a target vein without necessitating smartwatch movement, comprising:
a slider coupled to the base module via one or more rails underneath the base module, the slider further coupled to the base module by a linking cable that enables the processor to communicate with the antenna bands, and the slider configured to slide across the base module; and a bottom slider surface in which the at least one antenna band is fabricated.

9. The wearable health monitoring device of claim 8, wherein the at least one antenna band is flush with the bottom slider surface.

10. The wearable health monitoring device of claim 8, wherein when at least one antenna band is well-aligned over the target vein, the smartwatch receives constant notifications including stronger signals the more directly the antenna bands are over the target vein; wherein notifications include one or more of: visual alerts, voice messages, or specific vibrations.

11. The wearable health monitoring device of claim 8, wherein the antenna bands continuously or sequentially transmit and receive RF detection signals as programmed with the processor.

12. The wearable health monitoring device of claim 8, wherein the antenna bands are configured to individually activate a transmit antenna of one antenna band and a receive antenna of another antenna band.

13. The wearable health monitoring device of claim 8, wherein the base module includes a communications module for communicating with the smartwatch.

14. The wearable health monitoring device of claim 8, wherein the output information corresponds to one or more of blood glucose level, heart rate, blood pressure, and oxygen saturation.

15. A wearable health monitoring device for detachable attachment to a smartwatch, comprising:
at least one antenna band, including:
a plurality of transmit antenna configured to transmit radio frequency (RF) detection signals into a skin surface of a user; and
a plurality of receive antenna configured to receive responded RF detection signals that result from the RF detection signals transmitted into the user;
a base module configured for attachment to a bottom surface of the smartwatch, including:
a semiconductor chip configured to convert the responded RF detection signals into high-power RF signals;
an analog to digital converter (ADC) configured to receive the high-power RF signals and to convert the high-power RF signals into digital signals; and
a processor coupled with the semiconductor chip and configured to convert the digital signals into output information; and
an alignment feature assembly for positioning antenna bands over a target vein without necessitating smartwatch movement, comprising:
a panel, attached directly to the base module, including an array of antenna bands from the at least one antenna band on the bottom surface of the panel arranged in rows and columns.

16. The wearable health monitoring device of claim 15, wherein the at least one antenna band is flush with the bottom surface of the panel.

17. The wearable health monitoring device of claim 15, wherein the array of antenna bands are configured to sequentially activate and deactivate to confirm the presence of a target vein.

18. The wearable health monitoring device of claim 15, wherein the processor is configured to active the transmit antenna and the receive antenna of pre-configured groups of the antenna bands.

19. The wearable health monitoring device of claim 15, wherein the antenna bands continuously or sequentially transmit and receive RF detection signals as programmed with the processor.

20. The wearable health monitoring device of claim 15, wherein the base module includes a memory unit that stores output information in a database of segregated raw data samples and derived data.
